(19) United States
(12) Patent Application Publication
(10) Pub. No.: US 2022/0331203 A1
(43) Pub. Date: Oct. 20, 2022

(54) SYSTEMS AND APPROACHES FOR DRUG DELIVERY DEVICE RECONSTITUTION

(71) Applicant: AMGEN INC., Thousand Oak, CA (US)

(72) Inventor: Shawn Malloy, Cambridge, MA (US)

(21) Appl. No.: 17/763,677

(22) PCT Filed: Oct. 16, 2020
(86) PCT No.: PCT/US20/55873

§ 371 Date: Mar. 25, 2022

Related U.S. Application Data
(60) Provisional application No. 62/923,179, filed on Oct. 18, 2019.

(51) Int. Cl. A61J 1/20; A61M 5/14; A61M 5/142

(52) U.S. Cl. CPC A61J 1/2089; A61M 5/1409; A61M 5/14212; A61M 2005/1657

(57) ABSTRACT

Methods of preparing a drug for delivery and drug delivery systems for achieving the same are disclosed. A method of preparing a drug for delivery may include providing a diluent contained in a diluent container and providing a drug product contained within a drug product container. The method may further include fluidly connecting the diluent container and the drug product container. Additionally the method may include urging, via a pump, at least a portion of the diluent from the diluent container into the drug product container to at least partially reconstitute the drug product.

### CROSS-REFERENCE TO RELATED APPLICATION

The priority benefit of U.S. Provisional Application No. 62/923,179, filed Oct. 18, 2019, entitled “Systems And Approaches For Drug Delivery Device Reconstitution,” is hereby claimed and the entire contents thereof are incorporated herein by reference.

### FIELD OF DISCLOSURE

The present disclosure generally relates to drug delivery devices and, more particularly, to reconstitution approaches for drug delivery devices.

### BACKGROUND

Drugs are administered to treat a variety of conditions and diseases. Intravenous (“IV”) therapy is a drug dosing process that delivers drugs directly into a patient's vein using an infusion contained in a delivery container (e.g., a pliable bag). These drug dosings may be performed in a healthcare facility, or in some instances, at remote locations such as a patient's home. In certain applications, a drug product may be shipped to a healthcare facility (e.g., an inpatient facility, an outpatient facility, and/or a pharmacy) in a powdered or lyophilized form.

When reconstituting these drugs for administration, it is of particular importance to maintain a sterile environment so as to not taint, compromise the sterility of, or otherwise damage the quality of the drug. Additionally, some classes of drugs such as bi-specific T-cell engagers may require exceptionally accurate quantities of the drug product and/or other fluids required for dosing so as to prevent the drug product from becoming toxic. Oftentimes, the healthcare professional must prepare the drug by closely following a set of steps to ensure a sterile environment is maintained and that correct quantities of ingredients are added to the delivery container, including in certain instances according to a correct sequence. When reconstituting these drugs for administration, it may be desirable or necessary to utilize a diluent, such as by adding a diluent to a drug product vial. As a result of these various steps and requirements, the reconstitution process may be time-consuming, tedious, and may have an unacceptable or undesirable error rate.

The current process of reconstituting a lyophilized oncology product is often done either at the hospital or the specialty compounding pharmacy by a licensed pharmacist. The use of a hood is often required to perform reconstitution steps to provide a sterile working environmental which can be cumbersome for pharmacist given the complexity of the steps. In addition, this reconstitution process involves the use of multiple needles to withdraw/add sterile water for injection (WFI), saline and/or Intravenous Solution Stabilizer (IVSS) solutions. Typically, for relatively complex oncology products such as a Bi-specific T-cell Engager (BITE®) molecule (e.g. Blincyto®) prepared in an IV bag, a specified volume of WFI is added to reconstitute a lyophilized drug product contained in a vial via the use of a needle and syringe system. Then, the applicable volume of saline and IVSS solutions are added to an empty IV bag before the final reconstituted drug product is introduced. The overall process may take up to 5 needle and syringe systems, each of which carries manual labor time and exposure to a potential needle. Furthermore, the use of a hood during this complex preparation may introduce risks.

In addition, with the current regulatory requirements implemented by National Institute for Occupational Safety and Health (NIOSH), certain oncology products are included in the hazardous drug list which require the use of additional engineering controls such as Closed System Transfer Device (CSTD) as an additional means of protection. Also, regardless of whether a drug is on the NIOSH list, it may be advantageous to utilize a CSTD and/or other components/systems to minimize or avoid undesired release of fumes into the air or other exposures.

As described in more detail below, the present disclosure sets forth systems and methods for drug delivery device reconstitution embodying advantageous alternatives to existing systems and methods, and that may address one or more of the challenges or needs mentioned herein, as well as provide other benefits and advantages.

### SUMMARY

An aspect of the present disclosure provides a method of preparing a drug for delivery. The method may include: (a) providing a diluent contained in a diluent container; (b) providing a drug product contained within a drug product container; (c) fluidly connecting the diluent container and the drug product container; and (d) urging, via a pump, at least a portion of the diluent from the diluent container into the drug product container to at least partially reconstitute the drug product.

An additional aspect of the present disclosure provides a drug delivery system including a diluent container, a drug product container, at least one fluid path connector, and a pump. The diluent container may contain a diluent, and the drug product container may contain a drug product. The at least one fluid path may be configured to at least selectively fluidly connect the diluent container and the drug product container. The pump may be in working connection with the fluid path connector and configured to urge at least a portion of the diluent from the diluent container into the drug product container to at least partially reconstitute the drug product.

### BRIEF DESCRIPTION OF THE DRAWINGS

The above needs are at least partially met through provision of the systems and approaches for drug delivery device reconstitution described in the following detailed description, particularly when studied in conjunction with the drawings, wherein:

FIG. 1 illustrates exemplary drug delivery system components in accordance with various embodiments.

FIG. 2 illustrates a drug pump shown in FIG. 1 in an operational configuration, in accordance with various embodiments;

FIG. 3 illustrates an example drug delivery system in accordance with various embodiments;

FIG. 4 illustrates an example approach for preparing a drug delivery device using the system of FIG. 1 in accordance with various embodiments;

FIG. 5 illustrates an example usage configuration of a drug delivery system in accordance with various embodiments;

FIG. 6 illustrates an example drug delivery system in accordance with various embodiments; and

FIG. 7 illustrates an example drug delivery system in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

The present disclosure relates to a drug delivery device and a method of preparing a drug delivery device, generally including: a diluent container containing a diluent, a drug product container containing a drug product, a fluid path connector that at least selectively fluidly connects the diluent container and the drug product container, and a pump in working connection with (e.g., operably connected to) the fluid path connector and configured to urge at least a portion of the diluent from the diluent container into the drug product container to at least partially reconstitute the drug product. The present disclosure also may include a solution container containing a predetermined quantity of saline solution and a predetermined quantity of IV stabilizing solution (“IVSS”). In such a system, the pump is able to urge at least a portion of the predetermined quantity of saline solution and the predetermined quantity of IVSS from the solution container into the drug product container.

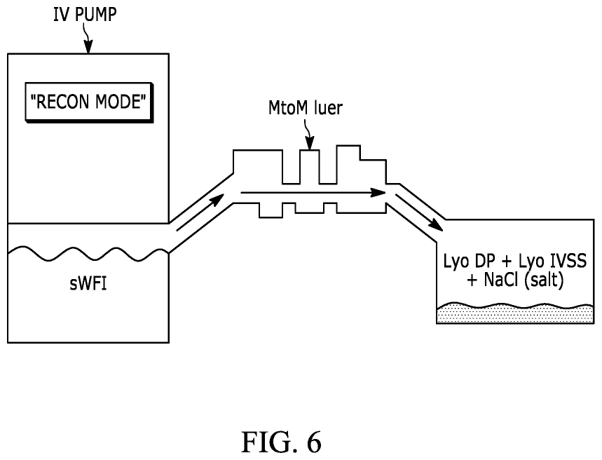

For example, a drug product can be bulk lyophilized and filled into cartridges that are typically used to administer with an IV pump. If needed the dehydrated forms of IVSS, NaCl, and any other components needed for the final administered solution can be bulk lyophilized and filled into the cassette for long term storage. As part of a kit for any dosing cassette, a complementary sterile water for injection (sWFI) cassette can be provided that will act as the diluent to reconstitute the product for administration. The reconstitution can be done in a simple and eloquent way, utilizing the administration pump itself to perform the reconstitution with no additional accessories. The sWFI cartridge may then be coupled with the IV pump, as seen in FIG. 6. For example, two cartridges are then connected via a simple luer adapter or an integrated connection in the cartridge. A simple button or mode setting for “Reconstitution Mode” can then be selected, which will direct the pump to empty the contents of the sWFI cartridge into the Lyo cartridge to reconstitute it, as seen in FIG. 6. After contents are emptied, the fully reconstituted cassette can then be hooked up to the pump and administer to the patient. This preparation may be able to be completed in a pharmacy or another setting such as a HCP's office or a patient's home.

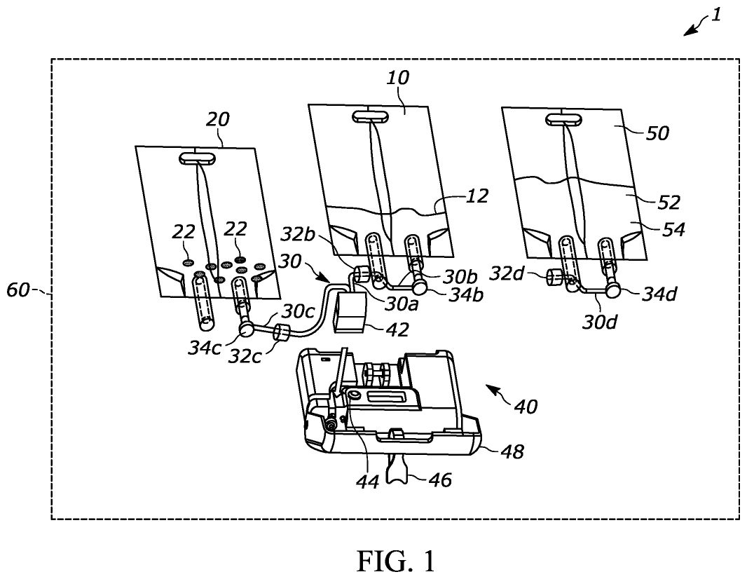

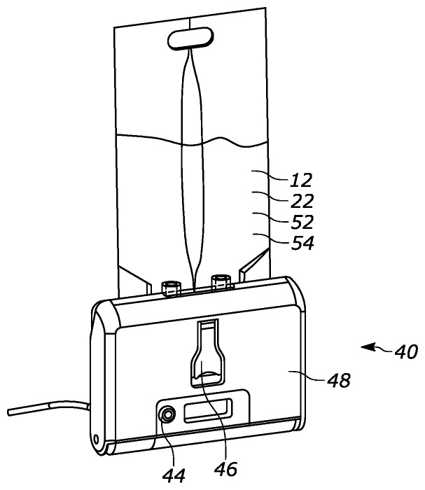

Turning to FIGS. 1-2, pursuant to these various embodiments, a drug delivery system 1 or kit and a corresponding method of preparing a drug delivery device using the drug delivery system 1 are provided. The drug delivery system 1 can be used by a healthcare professional, a caregiver, or patient to prepare a drug delivery device to be delivered to a patient. The drug delivery system 1 varies from conventional systems in that a number of the components included in the system 1 come prefilled and/or premixed in correct dosage quantities. As a result, preparation of the drug delivery device by the healthcare professional, caregiver, or patient is reduced while still ensuring correct quantities of ingredients are administered. The system 1 may be used to provide intravenous, subcutaneous, intra-arterial, intramuscular, and/or epidural delivery approaches. By using the system 1, patient anxiety and or confusion may be reduced due to reduced preparation complexity and wait times caused by the drug preparation process. Additionally, the system 1 may permit a health care provider, a pharmacist, a patient, and/or other individuals involved in preparing, providing, or using medication to have a more streamlined, predictable, and/or effective process for drug delivery. For example, the system 1 may reduce the amount of time a pharmacist spends preparing medication for use by a patient, reduce the number of steps a pharmacist must manually take to prepare medication for use by a patient, and/or improve the overall efficiency of the medication preparation process. As a more specific example, the system 1 may be particularly advantageous for use with medication preparation that involves several steps, such as adding a diluent and then adding a solution containing saline and/or IVSS, and/or medication that requires extensive preparation time.

The drug delivery system 1 shown in FIG. 1 includes, generally, a diluent container 10 containing a diluent 12, a drug product container 20 containing a drug product 22, a fluid path connector 30 that at least selectively fluidly connects the diluent container 10 and the drug product container 20, and a pump 40 in working connection with the fluid path connector 30 and configured to urge at least a portion of the diluent 12 from the diluent container 10 into the drug product container 20 to at least partially reconstitute the drug product 22. The drug delivery system 1 shown in FIG. 1 may also include a solution container 50 containing a predetermined quantity of saline solution 52 and a predetermined quantity of IV stabilizing solution (“IVSS”) 54. The predetermined quantity of saline solution 52 and the predetermined quantity of IVSS 54 shown in the figures are mixed to form a generally aqueous solution, but other configurations may also be suitable. In such a system, the pump 40 is able to urge at least a portion of the predetermined quantity of saline solution 52 and the predetermined quantity of IVSS 54 from the solution container into the drug product container.

The pump 40 shown in FIG. 1 may be a peristaltic pump, a positive displacement pump, or any other suitable type of pump that is workingly connected to the fluid path connector 30. For example, fluid path connector 30 may be a tube having a portion that is looped into a generally circular shape and the removable pump head 42 may be a peristaltic pump head having a tube portion and a rotating component that travels in a generally circular travel path and, along the way, pinches the tube portion and urging fluid to travel through the tube. In such a configuration, the removable pump head 42 is workingly connected to the fluid path connector 30 even though the fluid traveling through the tube does not directly contact the pump head 42 components. As another example, the pump may have components that directly contact the fluid traveling through the tube. As another example, any suitable pump may be used. Additionally, or alternatively, instead of a pump the system may utilize another configuration or process for mixing, such as negative pressure arrangement between the various containers that urges the diluent 12 into the drug product container 20.

The pump 40 may be activated automatically once one or more of the respective containers 10, 20, 50 are coupled with each other or the pump 40 may be activated by an activation button 44 or other suitable component. For example, the activation button 44 may be workingly connected to an internal controller and/or electro-mechanical components that operate the pump 40.

The fluid path connector 30 may include several different tubes, such as tube 30a that is coupled with the pump head 42, tube 30b that is fluidly coupled with the diluent container 10 via a stake connector 34b, a tube 30c fluidly connected with the drug product container 20 via a stake connector 34c, and/or a tube 30d fluidly connected with the solution container 50 via a stake connector 34d. The connectors 32b, 32c, 32d may be quick-connect sterile connectors with respective sub-components that selectively mate with each other while maintaining sterility or another desirable cleanliness standard. For example, the quick-connect sterile connectors may snap or twist or screw together; they may have sheathed or covered components that become unsheathed or uncovered upon connection; and/or they may have Luer Lock or modified Luer Lock configurations. During one exemplary operation, the diluent container 10 is selectively coupled with the drug product container 20 via the connectors 32b, 32c and the pump head 42 operates to urge diluent 12 from the diluent container 10 into interaction with the drug product 22 in the drug product container. Next, during another step in an exemplary operation, the diluent container 10 is disconnected from fluid connection with the pump head 42 via connector 32b and the solution container 50 is then fluidly coupled with the tube 30a via connector 32d. Then, the pump head is able to urge the saline solution 52 and IVSS 54 into the drug product container 20 so that the various components (drug product 22, diluent 12, saline solution 52, and IVSS 54) are sufficiently mixed and the combination is available for delivery to a patient. As a more specific example, once the drug product container 20 has a desired mixture of desired components, the drug product container 20 may be fluidly connected with the pump head 42 for delivery to a patient via an intravenous line, a port, a catheter, or other suitable drug delivery components.

Although the respective containers 10, 20, 50 shown in FIG. 1 are fluidly coupled with each other two at a time, but other suitable configurations may be used, such as if the tube 30d is fluidly connected with the tube 30b while the tube 30b is also fluidly connected to the tube 30c. For example, the tube 30d may be fluidly connected with the stake 34b such that the pump urges the diluent from the diluent container 10 until the diluent container 10 is empty or substantially empty and then vacuum forces from the pump 42 urge the contents of the solution container 50 through the various components of the fluid path connector 30 and into the drug product container 20.

Additionally, or alternatively, the direction that the respective components 12, 22, 52, 54 is drawn and the containers 10, 20, 50 in which the components are mixed may vary. For example, the diluent 12 may be urged into the drug product container 20 as discussed above and then the drug product 22/diluent 12 mixture may be urged into the solution container 50 for mixture with the solution 52 and IVSS 54. The connectors 32b, 32c, 32d permit flexible configurations for the user.

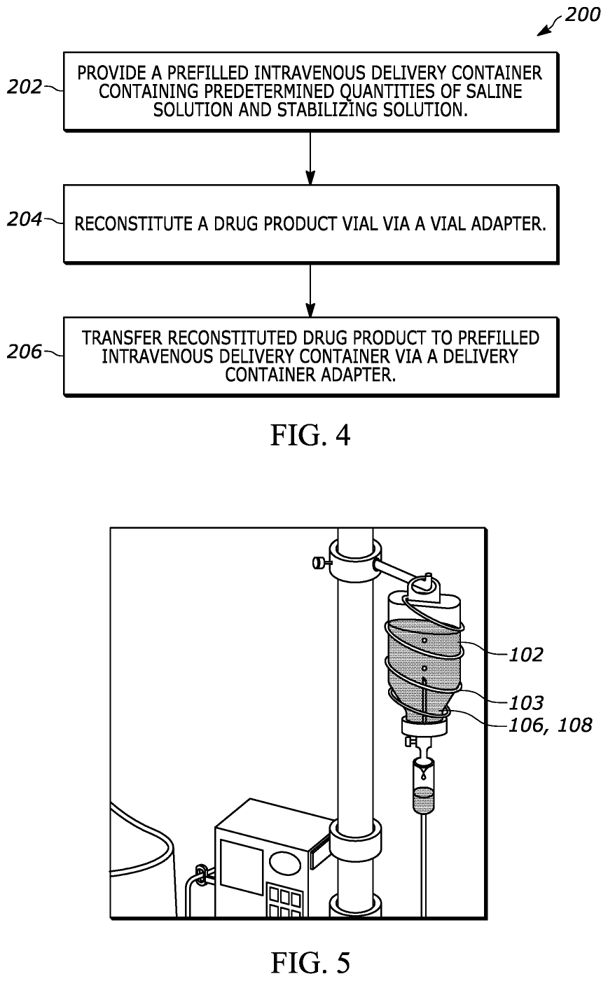

In some examples, the IVSS 54 may be provided as a percentage of an overall volume of solution. In these examples, suitable quantities of IVSS 54 may range between approximately 2% and approximately 15% (e.g., between approximately 1 mL in a 50 mL container and approximately 25 mL in a larger, 270 mL container; see FIG. 4 at step 202). The IVSS 54 can also act as a pretreating surfactant or a buffering component that prevents adsorption of the drug onto the walls of the container 50. For example, due to the highly potent nature of some drugs being administered, if the container is not sufficiently and properly coated with the IVSS 54, it may lead to an undesirable risk of drug molecules adhering or adsorbing to the inner walls of the container. In the event of adsorption of the drug onto the delivery container walls, the dosage of the drug may be adversely impacted. In such a situation, it may be desirable to utilize the exemplary steps discussed in the prior paragraph.

In some examples, the IVSS 54 may include polysorbate. In some examples, the IVSS 54 formulation may include approximately 1.25 M lysine monohydrocholoride, 25 mM citric acid monohydrate, 0.1% (w/v) polysorbate 80, and has a pH of approximately 7.0. In other examples, the IVSS 54 may include similar formulations, but also have a minimum of approximately 0.9% NaCl and approximately 0.001 to approximately 0.1% (w/v) polysorbate 80. It is appreciated that different BiTEs require different final percentages of IVSS 54 in the delivery container. This percentage may vary between approximately 0.5% to approximately 12% of the final volume in the delivery container. Further, citrate may increase the risk of glass delamination if filled in glass vials. In the event that citrate is necessary for drug product stabilization (determined on a per-product basis), the delivery containers may be constructed from crystal zenith (CZ) polymer or other plastic compositions. Other examples of ingredients for suitable IVSSs 54 are possible. Suitable IVSS 54 concentrations protect against protein-plastic interactions and/or surface adsorption, and more specifically, in the lower end of the concentration range where even minor losses may potentially change the effective dose. The below table illustrates example component concentrations for varying IVSS concentrations:

TABLE 1
Component Concentrations with Varying IVSS Concentrations
(top column units are (V/v) % of IVSS

| IVSS COMPONENTS | 0.5 | 1.0 | 2.0 | 4.0 | 6.0 | 8.0 | 10.0 | 12.0 |
|---|---|---|---|---|---|---|---|---|
| Lysine monohydrochloride | 0.00625 | 0.0125 | 0.025 | 0.05 | 0.075 | 0.1 | 0.125 | 0.15 |
| (M) | | | | | | | | |
| Citrate Monohydrate (M) | 0.000125 | 0.00025 | 0.0005 | 0.001 | 0.0015 | 0.002 | 0.0025 | 0.003 |
| Polysorbate 80 (% w/v) | 0.0005 | 0.001 | 0.002 | 0.004 | 0.006 | 0.008 | 0.01 | 0.012 |

By providing the components 12, 22, 52, 54 in containers that are selectively connectable, it is no longer necessary to prepare a needle and syringe assembly to inject one component into another container, to ensure that this prepared needle and syringe assembly is sterilized, and/or to ensure a correct volume or amounts of components are added together.

Some conventional systems may provide delivery containers having saline solution overfill, where more saline solution is provided in the delivery container than what is needed for dosage. In these systems, it may be necessary to remove a volume of the saline solution prior to preparing the drug dosage, which may require preparing a sterile withdrawal tool (e.g., a needle and syringe assembly) and carefully extracting an accurate amount of saline solution. Conversely, the disclosed system 1 additionally eliminates this process, as the containers are prefilled with the required quantity of components. Additionally, the risk of a needle sticking due to the transfer of the components may also be reduced or mitigated.

Additionally, many or all of the above described steps may be automated or semi-automated or reduced in time/scope, thereby potentially saving time and effort for the persons preparing and/or using the drug.

As discussed above, the drug product container 20 contains a predetermined quantity of drug product 22 or active pharmaceutical ingredient (“API”) (e.g., between approximately 2 mcg and approximately 100 mcg), depending on the BiTE® and container size, which, in the illustrated example, is in powdered form (i.e., lyophilized) requiring reconstitution. In other examples, the drug product 22 may be in liquid form and may not require reconstitution. Nonetheless, the system 1 includes an accurate quantity of drug product 22, and thus does not require the need to add additional quantities thereto in a sterile environment. In some examples, the API may be in the form of a half-life extended (“HLE”) BiTE® and/or an IV-admin monoclonal antibody (“mAbs) as desired. These HLE BiTEs include an antibody Fc region that advantageously provides different drug properties such as longer and extended half-lives. Accordingly, such APIs may be preferred due to their ability to maintain protective levels in the patient for relatively longer periods of time. Nonetheless, in other examples, the API may be in the form of a canonical-BiTE that is to be administered in a professional healthcare environment.

In some embodiments, the drug delivery system 1 may have an integrated reconstitution subsystem onboard to dilute a lyophilized drug into a liquid form. In certain such embodiments, a diluent reservoir may be included for storing a diluent solution and a lyophilized reservoir may be included storing a lyophilized compound separate from the diluent solution. Furthermore, a fluid drive mechanism may be included for mixing the diluent solution in the diluent reservoir with the lyophilized compound in the lyophilized reservoir. In some embodiments, the fluid drive mechanism may transfer the diluent solution from the diluent reservoir into the lyophilized reservoir and/or provide any circulation and/or agitation needed to achieve full reconstitution. In some embodiments, an additional final reconstituted drug reservoir may be included and serve as a delivery reservoir from which the reconstituted drug is discharged into the patient; whereas, in other embodiments, the lyophilized reservoir may serve as the delivery reservoir. While the reconstitution subsystem may be physically integrated into the drug delivery system 1 in certain embodiments, in other embodiments the reconstitution subsystem may constitute a separate unit which is in fluid communication with the drug delivery system 1. Having a separate unit may simplify the reconstitution process for healthcare providers in certain cases.

The drug product container 20 may be in the form of an IV bag, a vial, a prefilled syringe, or similar container that includes a reconstitution container body defining an inner volume. The inner volume may be sterile. In some approaches, the reconstitution container adapter may also be a CSTD (or, in examples where the prefilled reconstitution container is in the form of a syringe, the container adapter may be a needle) that mates, engages, and/or couples to the vial adapter. Additionally or alternatively, the drug product 22 can be bulk lyophilized and filled into a cartridge or container that is typically used to administer with an IV pump. If needed the dehydrated forms of IVSS, NaCl, and any other components needed for the final administered solution can be bulk lyophilized and filled into the cassette for long term storage.

The prefilled diluent container 10 contains a predetermined quantity of diluent 12 (e.g., preservative-free water for injection or “WFI”) (e.g., between approximately 0.5 mL and approximately 10 mL) to be added to the prefilled drug product container 20 for reconstitution of the drug product 22. In some examples, a benzyl alcohol preserved (or any other preservative) WFI may be used.

As previously noted, in some examples, the prefilled drug product container may be in the form of a prefilled syringe that contains the drug product. In these examples the drug product may be in the form of a liquid BiTE® formulation used in conjunction with a monoclonal antibody (mAb), In these examples, the drug product may be directly added to the delivery container without the use of a vial adapter system (such as the above-mentioned CSTDs) where more traditional needle-syringe injection/delivery into the container is preferred, which may advantageously simplify and/or improve supply chain and manufacturing control, and may further allow for more compact commercial packaging that takes up less space in storage systems at healthcare facilities. In these examples, the prefilled drug product vial may or may not need to be reconstituted prior to transferring the drug product to the delivery container.

The system 1 may be distributed and/or sold as a common kit packaging 60, but other suitable distribution/packaging is suitable. The drug product may be in the form of a half-life extended bispecific T cell engager (BITE®), but other drug products are suitable. The diluent 12 include water for injection (“WFI”), but other diluents may be suitable. The containers 10, 20, 50 may be pliable (e.g., flexible) bags, such as IV bags, but other containers may be suitable. In some examples, one or more of the containers 10, 20, 50 is in the form of an IV drip bag constructed from a plastic or other material, e.g., 250 mL 0.9% Sodium Chloride IV bag constructed of a suitable material such as polyolefin, non-DEHP (diethylhexl phthalate), PVC, polyurethane, or EVA (ethylene vinyl acetate) and can be filled to a volume of approximately 270 mL to account for potential moisture loss over long-term storage.

During some or all of the above steps, the contents of a container may then be gently stirred, swirled, and/or inverted to mix the ingredients, thereby forming a desired mixture. Similarly, the mixtures may be visually inspected for imperfections and/or to ensure adequate mixing has occurred.

Once the drug product 22 and other components are mixed as desired, the drug product container 20 (or whatever container is holding the mixed drug product 22 and other components) may be delivered to a patient utilizing the pump 40. For example, the same pump head 42 may be used but with one end connected to the container 10 and another end connected to a patient. Alternatively, a new, fresh pump head 42 may be used for this next step. The pump head 42 may be disposable or reusable. The remainder of the pump 40 likewise may be reusable or disposable (preferable reusable for environmental and cost advantages). The pump may also have different modes, such as a “reconstitution mode” where the pump is operating under one set of parameters and a “delivery mode” where the pump is operating under another set of parameters. Alternatively, or additionally, the pump may operate in different modes or speeds or other parameters via controls on the pump itself or via controls on another device such as a wirelessly-paired smartphone or other suitable device.

The pump 40 may include a door 48 and a lock 46 that facilitate removal and insertion of a pump head 42 component and/or for operation safety reasons. The pump 40 may be configured such that it does not operate unless the door 48 and lock 46 are in desired positions.

FIG. 2 shows the pump 40 in an exemplary drug delivery mode, where the drug product 22 is dissolved and the mixed components 12, 22, 52, 54 are uniformly distributed throughout the container 20. The reconstitution mode may look similar or the same as the configuration shown in FIG. 2, but with additional container(s) coupled with each other.

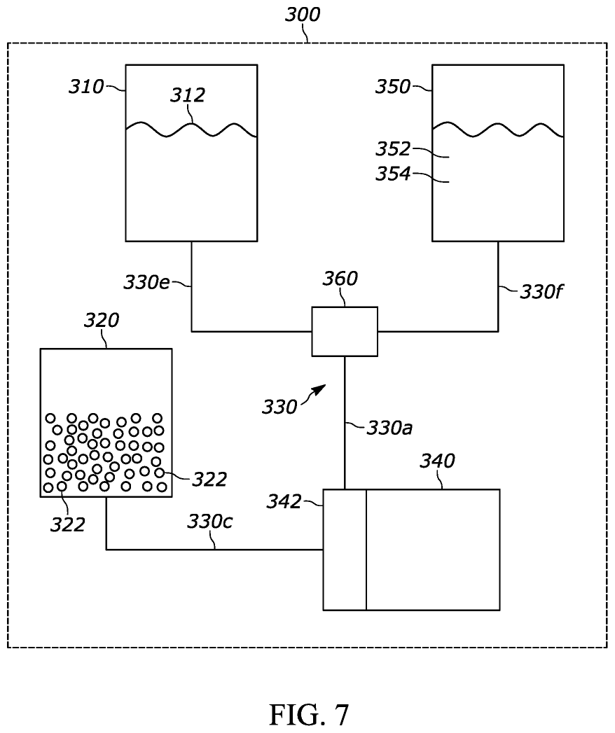

The fluid path connector 30 shown in FIG. 1 may be connected to a single one of the diluent container 10 and the solution container 50 at any given time depending on which of these containers is currently transferring fluid to the drug product container 20. The drug delivery system 300 depicted in FIG. 7 includes many similar or identical components as those shown in FIG. 1 and described above, except that the system 300 additionally includes a valve 360. As described in more detail below, the valve 330 may allow for the fluid path connector 330 to remain connected to both the diluent container 310 and the solution container 350 throughout the reconstitution process. The elements of the drug delivery system 300 not described in more detail below may have similar or identical configurations, functions, and/or structure as the correspondingly numbered elements described above with respect to the drug delivery system 1 shown in FIG. 1.

The valve 360 may be configured to selectively fluid connect one of the diluent container 310 and the solution container 350 to the drug product container 320. As an example, the valve 360 may have at least two inlets or ports fluidly connected with, respectively, the diluent container 310 and the solution container 350 via, respectively, a tube 330e and a tube 330f. The valve 360 may additionally have an at least one outlet or port fluidly connected with the pump head 342 via the tube 330a. As a more specific example, the valve 360 may include a moveable or actuatable component which depending on its position or state opens a passageway between one of the two inlets and the outlet while closing a passageway between the other one of the two inlets and the outlet, and vice versa. As an even more specific example, the valve 360 may be a 3-way valve including, for example, a 3-way ball valve having an L-shaped fluid passageway inside of, for example, a rotor. As another example, the valve 360 may include an electronically controllable element such as a solenoid for selectively fluidly connecting one of the diluent container 310 and the solution container 350 to the drug product container 320. As another example, the valve 360 may incorporate a hydrophilic filter. The hydrophilic filter may be configured such that upon depletion of fluid from one of the diluent container 310 and the solution container 350 the hydrophilic filter may force or direct withdrawal of fluid from the other one of the diluent container 310 and the solution container 350. As a more specific example, the hydrophilic filter may be configured to permit the passage of fluid but prevent the passage of gas such that upon depletion of fluid from one of the diluent container 310 and the solution container 350 the hydrophilic filter may close off a pathway to the depleted container and, in at least some configurations, may cause a suction force from the pump 340 to be directed to withdrawing fluid from the other one of the diluent container 310 and the solution container 350.

As shown in FIG. 7, the valve 360 may be a component that is separate from the pump 340 and the pump head 342. In other embodiments, the valve 360 may be integrated into the pump 340 and/or the pump head 342.

During one exemplary reconstitution process, the valve 360 may be configured to fluidly connect the diluent container 310 to the drug product container 320, and the pump head 342 may be driven by the pump 340 to urge the diluent 312 from the diluent container 310 into interaction with the drug product 322 in the drug product container 320. Next, during another step in an exemplary operation, the valve 360 may be configured to fluidly disconnect the diluent container from the drug product container 320 and instead fluidly connect the solution container 350 to the drug product container 320. Subsequently, the pump head 342 may be driven by the pump 340 to urge the saline solution 352 and the IVSS 354 into the drug product container 320 so that the various components (e.g., the drug product 322, diluent 312, saline solution 352, and IVSS 354) are sufficiently mixed and the combination is available for delivery to a patient. Once the drug product container 320 has a desired mixture of desired components, the drug product container 320 may be fluidly connected with the pump head 342 for delivery to a patient via an intravenous line, a port, a catheter, or other suitable drug delivery component(s). In some embodiments, these drug delivery components(s) may be connected to one of the ports of the valve 360 used for connecting the valve 360 with the diluent container 310 or the solution container 350 during the reconstitution process or another port of the valve 360.

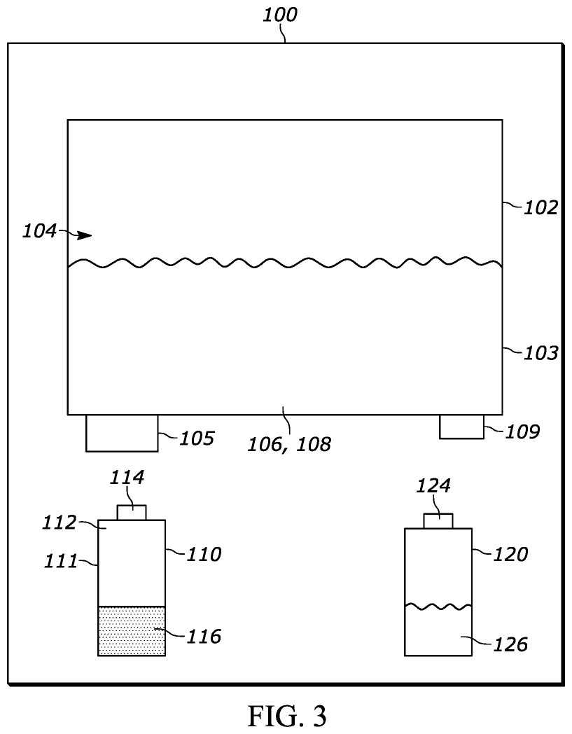

Turning to FIGS. 3-5, pursuant to various embodiments, a drug delivery system 100 or kit and a corresponding method 200 of preparing a drug delivery device using the drug delivery system 100 are provided. Many or all of the characteristics of the system 100 may be utilized with many or all of the characteristics of the system 1. Additionally, many or all of the characteristics of the system 1 may be utilized with many or all of the characteristics of the system 100.

The drug delivery system 100 can be used by a healthcare professional, a caregiver, or patient to prepare a drug delivery device to be delivered to a patient. The drug delivery system 100 varies from conventional systems in that a number of the components included in the system 100 come prefilled and/or premixed in correct dosage quantities. As a result, preparation of the drug delivery device by the healthcare professional, caregiver, or patient is reduced while still ensuring correct quantities of ingredients are administered. The system 100 may be used to provide intravenous, subcutaneous, intra-arterial, intramuscular, and/or epidural delivery approaches. By using the system 100, patient anxiety and or confusion may be reduced due to reduced preparation complexity and wait times caused by the drug preparation process.

Generally, and as illustrated in FIG. 3, the drug delivery system 100 includes a prefilled delivery container 102, a prefilled drug product vial 110, and a prefilled reconstitution container 120. More specifically, the prefilled delivery container 102 includes a container body 103 defining an inner volume 104, a delivery container adapter 105, and an IV line outlet 109 that allows tubing to be coupled thereto in order to deliver the prescribed drug. In some examples, the prefilled delivery container 102 is in the form of an IV drip bag constructed from a plastic or other material, e.g., 250 mL 0.9% Sodium Chloride IV bag constructed of a suitable material such as polyolefin, non-DEHP (diethylhexl phthalate), PVC, polyurethane, or EVA (ethylene vinyl acetate) and can be filled to a volume of approximately 270 mL to account for potential moisture loss over long-term storage. Other examples of suitable delivery containers are possible such as, for example, a glass bottle or container (see, e.g., FIG. 5). Example suitable prefilled delivery containers 102 are described in U.S. Appln. No. 62/804,447, filed on Feb. 12, 2019 and U.S. Appln. No. 62/877,286 filed on Jul. 22, 2019, the contents of each of which are incorporated by reference in their entirety.

The delivery container adapter 105 may be a closed system transfer device (“CSTD”) that allows for transfer of the drug and/or fluids into the container body 103. Example CSTD devices may include the OnGuard CSTD provided by B. Braun Medical Inc, BD PhaSeal CSTD components, Equashield CSTD, Codon CSTD, and the like. Further, non-closed system transfer devices may be used such as West Pharmaceuticals vial and bag adapters. Other examples are possible. The prefilled delivery container 102 may include any number of delivery container adapters 105 having different specifications (e.g., port sizes) to accommodate the use of different drug product vials 110.

The prefilled delivery container 102 contains a predetermined quantity (e.g., a volume) of excipient solution. For example, the prefilled delivery container 102 can include a predetermined quantity of a saline solution 108 (e.g., between approximately 50 mL and 500 mL of 0.9% Sodium Chloride, and preferably, approximately 110 mL or approximately 270 mL, depending on the size of the container) and a predetermined quantity of an IV stabilizing solution (“IVSS”) 106. In some examples, the IVSS 106 may be provided as a percentage of an overall volume of solution. In these examples, suitable quantities of IVSS 106 may range between approximately 2% and approximately 15% (e.g., between approximately 1 mL in a 50 mL container 102 and approximately 25 mL in a larger, 270 mL container; see FIG. 4 at step 202). In some examples, the prefilled delivery container 102 may have a total volume of approximately 270 mL. The IVSS 106 can also act as a pretreating surfactant or a buffering component that prevents adsorption of the drug onto the walls of the container 102. For example, due to the highly potent nature of some drugs being administered, if the container 102 is not sufficiently and properly coated with the IVSS 106, it may lead to an undesirable risk of drug molecules adhering or adsorbing to the inner walls of the container 102. In the event of adsorption of the drug onto the delivery container walls 102, the dosage of the drug may be adversely impacted. In some examples, the IVSS 106 may include polysorbate 80. In some examples, the IVSS 106 formulation may include approximately 1.25 M lysine monohydrocholoride, 25 mM citric acid monohydrate, 0.1% (w/v) polysorbate 80, and has a pH of approximately 7.0. In other examples, the IVSS 106 may include similar formulations, but also have a minimum of approximately 0.9% NaCl and approximately 0.001 to approximately 0.1% (w/v) polysorbate 80. It is appreciated that different BiTEs require different final percentages of IVSS 106 in the delivery container 102. This percentage may vary between approximately 0.5% to approximately 12% of the final volume in the delivery container 102. Further, citrate may increase the risk of glass delamination if filled in glass vials. In the event that citrate is necessary for drug product stabilization (determined on a per-product basis), the delivery container 102 may be constructed from CZ or other plastic compositions. Other examples of ingredients for suitable IVSSs 106 are possible. Suitable IVSS 106 concentrations protect against protein-plastic interactions and/or surface adsorption, and more specifically, in the lower end of the concentration range where even minor losses may potentially change the effective dose. The below table illustrates example component concentrations for varying IVSS concentrations:

TABLE 1
Component Concentrations with Varying IVSS Concentrations
(top column units are (V/v) % of IVSS

| IVSS COMPONENTS | 0.5 | 1.0 | 2.0 | 4.0 | 6.0 | 8.0 | 10.0 | 12.0 |
|---|---|---|---|---|---|---|---|---|
| Lysine monohydrochloride | 0.00625 | 0.0125 | 0.025 | 0.05 | 0.075 | 0.1 | 0.125 | 0.15 |
| (M) | | | | | | | | |
| Citrate Monohydrate (M) | 0.000125 | 0.00025 | 0.0005 | 0.001 | 0.0015 | 0.002 | 0.0025 | 0.003 |
| Polysorbate 80 (% w/v) | 0.0005 | 0.001 | 0.002 | 0.004 | 0.006 | 0.008 | 0.01 | 0.012 |

By providing the prefilled IVSS 106 in the delivery container 102, the overall footprint of the system 100 is reduced, as separate containers used to contain the IVSS 106 are no longer needed. Additionally, it is no longer necessary to prepare a needle and syringe assembly to inject the IVSS 106 into the delivery container, to ensure that this prepared needle and syringe assembly is sterilized, and/or to ensure a correct volume of IVSS is added to the container 102.

Some conventional systems may provide delivery containers having saline solution 108 overfill, where more saline solution 108 is provided in the delivery container 102 than what is needed for dosage. In these systems, it may be necessary to remove a volume of the saline solution 108 prior to preparing the drug dosage, which may require preparing a sterile withdrawal tool (e.g., a needle and syringe assembly) and carefully extracting an accurate amount of saline solution 108. Conversely, the disclosed system 100 additionally eliminates this process, as the delivery container 102 comes prefilled with the required quantity of saline solution 108. Additionally, the risk of a needle sticking due to the transfer of the IVSS 106 into the container 102 and/or the transfer of the saline solution 108 out of the container 102 may also be reduced or mitigated.

The prefilled drug product vial or syringe 110 includes a vial body 111 defining an inner volume 112 and a vial adapter 114. The inner volume 112 may be sterile. In some approaches, the vial adapter 114 may also be a CSTD that mates, engages, and/or couples to the delivery container adapter 105. As with the prefilled drug delivery container 102, the inner volume 112 of the prefilled drug product vial 110 contains a predetermined quantity of drug product or active pharmaceutical ingredient (“API”) 116 (e.g., between approximately 2 mcg and approximately 100 mcg), depending on the BiTE® and vial size, which, in the illustrated example, is in powdered form (i.e., lyophilized) requiring reconstitution. In other examples, the drug product 116 may be in liquid form and may not require reconstitution. Nonetheless, the system 100 includes an accurate quantity of drug product 116, and thus does not require the need to add additional quantities thereto in a sterile environment. In some examples, the API may be in the form of a half-life extended (“HLE”) BiTE® and/or an IV-admin monoclonal antibody (“mAbs) as desired. These HLE BiTEs include an antibody Fc region that advantageously provides different drug properties such as longer and extended half-lives. Accordingly, such APIs may be preferred due to their ability to maintain protective levels in the patient for relatively longer periods of time. Nonetheless, in other examples, the API may be in the form of a canonical-BiTE that is to be administered in a professional healthcare environment.

The prefilled reconstitution container 120 may be in the form of a vial, a prefilled syringe, or similar container that includes a reconstitution container body 121 defining an inner volume 122 and a reconstitution container adapter 124. The inner volume 122 may be sterile. In some approaches, the reconstitution container adapter 124 may also be a CSTD (or, in examples where the prefilled reconstitution container 120 is in the form of a syringe, the container adapter 124 may be a needle) that mates, engages, and/or couples to the vial adapter 114. As with the prefilled drug delivery container 102 and the prefilled drug product vial 110, the prefilled reconstitution container 120 contains a predetermined quantity of diluent (e.g., preservative-free water for injection or “WFI”) 126 (e.g., between approximately 0.5 mL and approximately 10 mL) to be added to the prefilled drug product vial 110 for reconstitution of the drug product 116. In some examples, a benzyl alcohol preserved (or any other preservative) WFI may be used.

More specifically, the drug product 116 is reconstituted prior to addition into the delivery container 102 by mating the vial adapter 114 of the prefilled drug product vial 110 to the reconstitution container adapter 124 of the prefilled reconstitution container 120 and transferring the diluent 124 into the drug product vial 110 (see FIG. 4 at step 204). The contents may then be gently stirred, swirled, and/or inverted to mix the ingredients, thereby forming a mixed drug product. The reconstituted drug product vial 110 may then be visually inspected for imperfections and/or to ensure adequate mixing has occurred.

As previously noted, in some examples, the prefilled drug product vial 110 may be in the form of a prefilled syringe that contains the drug product 116. In these examples the drug product 116 may be in the form of a liquid BiTE® formulation used in conjunction with a monoclonal antibody (mAb), In these examples, the drug product 116 may be directly added to the delivery container 102 without the use of a vial adapter system (such as the above-mentioned CSTDs) where more traditional needle-syringe injection/delivery into the container 102 is preferred, which may advantageously simplify and/or improve supply chain and manufacturing control, and may further allow for more compact commercial packaging that takes up less space in storage systems at healthcare facilities. In these examples, the prefilled drug product vial 110 may or may not need to be reconstituted prior to transferring the drug product 116 to the delivery container 102.

The reconstituted drug contained in the prefilled drug vial 110 may then be transferred into the drug delivery container 102 by mating the vial adapter 114 of the prefilled drug product vial 110 to the delivery container adapter 105 of the delivery container 102 (see FIG. 4 at step 206). As a result, this transfer of the reconstituted drug into the delivery container 102 may be performed quickly (thus greatly reducing preparation times) and safely due to the lack of withdrawal assemblies (e.g., a luer lock needle and syringe mechanism). The systems described herein avoid and/or eliminate the potential occurrence of needle sticking and/or spills due to over-pressurizing of the vial. Additionally, contamination is mitigated due to the use of closed system transfer devices, whereas conventional assemblies use components that are open to the environment and thus can be subject to contamination.

The drug delivery system 100 may include any number of additional and/or optional features or alternatives. For example, any one or ones of the delivery container adapter 105, the vial adapter 114, or the reconstitution container adapter 124 may be in the form of ports or coupling mechanisms coupled to the prefilled delivery container 102, the prefilled drug product vial 110, and the reconstitution container 120, respectively. These ports may in turn be coupled to a CSTD device to allow for flow between the desired containers. Accordingly, CSTD devices having suitable coupling mechanism dimensions may be included in the system 100.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 146B7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40specific antibodies, peptibodies, related proteins, and the like, including but not limited to proteins that bind OX40L and/or other ligands of the OX40 receptor; Activase® (alteplase, tPA); Aranesp® (darbepoetin alfa); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4ß7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Humatrope® (somatropin, Human Growth Hormone); Humira® (adalimumab); Vectibix® (panitumumab), Xgeva® (denosumab), Prolia® (denosumab), Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP Ilb/Ilia receptor monoclonal antibody); Actemra® (anti-IL6 Receptor mAb); Avastin® (bevacizumab), HuMax-CD4 (zanolimumab); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 146B7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MY0-029); anti-GM-CSF Receptor mAb (CAM-3001); anti-HepC mAb (HuMax HepC); anti-IFNα mAb (MEDI-545, MDX-1103); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFß mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, or BPS 804 (Novartis) and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) antibodies such as but not limited to half-life extended BiTEs that include an antibody Fc region, BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A method of preparing a drug for delivery, the method comprising:
providing a diluent contained in a diluent container;
providing a drug product contained within a drug product container;
fluidly connecting the diluent container and the drug product container; and
urging, via a pump, at least a portion of the diluent from the diluent container into the drug product container to at least partially reconstitute the drug product.

2. The method of claim 1, further comprising activating the pump.

3. The method of claim 1, further comprising providing a predetermined quantity of saline solution and a predetermined quantity of IV stabilizing solution (“IVSS”) contained within a solution container.

4. The method of claim 3, further comprising urging, via the pump, at least a portion of the predetermined quantity of saline solution and the predetermined quantity of IVSS from the solution container into the drug product container.

5. The method of claim 3, further comprising providing a valve configured to selectively fluidly connect one of the diluent container and the solution container to the drug product container.

6. The method of claim 5, further comprising:
configuring the valve to fluidly connect one of the diluent container and the solution container to the drug product container; and
urging, via the pump, into the drug product container one of at least a portion of the diluent from the diluent container and at least a portion of the predetermined quantity of saline solution and the predetermined quantity of IVSS from the solution container.

7. The method of claim 6, further comprising:
configuring the valve to fluidly connect the other one of the diluent container and the solution container to the drug product container; and
urging, via the pump, into the drug product container the other one of the at least a portion of the diluent from the diluent container and the at least a portion of the predetermined quantity of saline solution and the predetermined quantity of IVSS from the solution container.

8. The method of claim 5, wherein the valve comprises a hydrophilic filter.

9. The method of claim 1, wherein at least one of the following:
(a) the diluent comprises water for injection (“WFI”),
(b) the diluent container is a pliable bag,
(c) the drug product container is a pliable bag,
(d) the solution container is a pliable bag,
(e) the IVSS comprises a pretreating surfactant or polysorbate 80, or
(f) the predetermined quantity of diluent is between approximately 0.5 mL and approximately 10 mL.

10-12. (canceled)

13. The method of claim 1, further comprising at least one of (a) fluidly coupling the diluent container to the drug product container via a sterile connector, or (b) fluidly coupling the solution container to the drug product container via a sterile connector.

14-16. (canceled)

17. The method of claim 3, wherein at least one of (a) the predetermined quantity of saline solution is between approximately 50 mL and approximately 500 mL, or (b) the predetermined quantity of IVSS is between approximately 1 mL and approximately 30 mL.

18. (canceled)

19. The method of claim 1, further comprising removing the drug product container and the diluent container from a common kit packaging.

20. The method of claim 1, wherein the drug product is in the form of a bispecific T cell engager (BiTE®), and optionally the BiTE® is a half-life extended (HLE) BiTE®.

21. (canceled)

22. A drug delivery system comprising:
a diluent container containing a diluent;
a drug product container containing a drug product;
at least one fluid path connector configured to at least selectively fluidly connect the diluent container and the drug product container; and
a pump in working connection with the fluid path connector and configured to urge at least a portion of the diluent from the diluent container into the drug product container to at least partially reconstitute the drug product.

23. The drug delivery system of claim 22, further comprising a solution container containing a predetermined quantity of saline solution and a predetermined quantity of IV stabilizing solution (“IVSS”).

24. The drug delivery system of claim 23, wherein the pump is configured to urge at least a portion of the predetermined quantity of saline solution and the predetermined quantity of IVSS from the solution container into the drug product container.

25. The drug delivery system of claim 23, wherein the at least one fluid path connector comprises a valve configured to selectively fluidly connect one of the diluent container and the solution container to the drug product container, wherein the valve optionally comprises a hydrophilic filter.

26. (canceled)

27. The drug delivery system of claim 22, wherein at least one of (a) the pump is a peristaltic pump, or (b).

28. The drug delivery system of claim 22, wherein the diluent container, the drug product container, and the pump are from a common kit packaging, wherein the common kit packaging optionally further includes the solution container and the at least one fluid path connector.

29. (canceled)

30. The drug delivery system of claim 22, wherein the drug product is in the form of a bispecific T cell engager (BiTE®), and optionally the BiTE® is a half-life extended (HLE) BiTE®.

31. (canceled)
